(19) United States
(12) Patent Application Publication
(10) Pub. No.: US 2001/0036646 A1
(43) Pub. Date: Nov. 1, 2001

(54) Mono- and poly-clonal anti-body-based macro- and micro-array technology to measure antibodies, antigens and small molecules

(76) Inventor: Gusheng Zhou, Northridge, CA (US)

Correspondence Address: David and Raymond Patent Group, 1050 Oakdale Lane, Arcadia, CA, 91006, US

(21) Appl. No.: 09/827,696

(22) Filed: Apr. 6, 2001

Related U.S. Application Data
(60) Provisional application No. 60/195,274, filed on Apr. 7, 2000.

(51) Int. Cl. G01N 33/537; G01N 33/543

(52) U.S. Cl. 435/7.93

(57) ABSTRACT

A process utilizing macro and micro array technology for measuring antibodies, antigens and small molecules by using mono- and poly-clonal antibody-based macro- and micro-array technology, includes the step of creating a reactant having an antibody; adding a test solution; washing out unbound antigens; adding a second antibody, washing out unbound conjugates; adding a detecting agent, and detecting a substrate. Therefore the macro- and micro-array technology is adapted to measure the existence of antibodies, antigens and small molecules.

### CROSS REFERENCE

This is a regular application of a provisional application, application Ser. No. 60/195,274, filed on Apr. 7, 2000.

### FIELD OF INVENTION

The present invention relates to method of making an identity card, and more particularly to a method for making a secured personal identity card and procedures for validation and obtaining secure personal information, including medical and/or DNA data, which can precisely distinguish an individual's identification and securely protect the privacy of the individual.

### BACKGROUND OF THE PRESENT INVENTION

Until very recently, biologists have studied molecules one at a time. Biology is so complex that one needs tools to study a multiplicity of molecules, including DNA, RNA and protein simultaneously. It is suggested that we are entering a new phase of biology where information is no longer limiting and integration of different technologies is required to attack the big problems of biology.

Though most cells in our bodies contain the same genes, not all of the genes are used in each cell. Some genes are turned on, or “expressed” when needed. Many genes are used to specify features unique to each type of cell. Liver cells, for example, express genes for enzymes that detoxify poisons, while pancreas cells express genes for making insulin. To know how cells achieve specialization, scientists need a way to identify which genes each type of cell expresses. The messenger RNA (“mRNA”) levels sensitively reflect the state of the cell. We should be able to monitor the mRNA expression level of all genes simultaneously. Recent technological advances in DNA micro-array (bio-chip) augur well for the eventually feasibility of this goal.

However, the functional molecules of biology are proteins produced from mRNA. Genes represent only the information banks for protein specification. Research at the protein level has lagged behind DNA- and RNA-based approaches due to the complexity of the technology required to separate, analyze and identify the thousands of proteins encoded by a typical genome. Until now, the tools necessary to study proteins have been limited in speed, sensitivity, accuracy and information content.

Proteomics, in essence, is the study of protein properties on a large scale to obtain a global, integrated view of disease processes, cellular processes, and network at the protein level. Proteome analysis should provide data on when, whether, sometimes where a predicated gene product is actually translated, its relative concentration compared with other gene-products, as well as the level and type of post transitional modification such as phosphorylation and glycosylation.

For proteomics to be widely adopted, a robust technology must be established that allows the large-scale discovery research needed for protein science. Proteome analysis is visualized most commonly up to date by 2-D gel electrophoresis and analyzed by matrix assisted laser desorption/ionization time-of-flight (MALDI/TOF) and tandem mass spectrometers. However, many researchers have expressed reservations about 2-D technology as a practical possibility in large-scale proteomics.

The invention of DNA micro-array technology has created a data boom in the pharmaceutical industry and research field. Tens of thousands of genes can now be screened on one array. Experiments involving gene expression, compound screening and toxicology can yield hundreds of thousands of data points in one experiment.

### SUMMARY OF THE PRESENT INVENTION

Accordingly, a main object of the present invention, is the use of monoclonal micro-arrays to measure antibodies, antigens and small molecules.

Another object of the present invention is the use of poly-clonal micro-arrays to measure antibodies, antigens and small molecules.

Another object of the present invention is the use of monoclonal macro-arrays to measure antibodies, antigens and small molecules.

Another object of the present invention is the use of poly-clonal macro-arrays to measure antibodies, antigens and small molecules.

Another object of the present invention is the use of micro- and poly-clonal micro- and macro-arrays to measure antibodies, antigens and small molecules.

### DETAILED DESCRIPTION OF THE PREFERRED EMBODIMENT

DNA molecules representing many genes are placed in discrete spots on a microscopic slide. This is called a DNA micro-array. Large arrays, termed “macro-array,” can be generated on filter (membrane) that is even easier to manipulate and can be used by any kind of research laboratories and hospitals. Hundreds and thousands of individual genes can be spotted on a single square inch slide or a couple of square inches membrane. Messenger RNA, the working copies of genes within cells, is purified from cells of a particular type. The RNA are then “labeled” by attaching a fluorescent dye or chemiluscent enzymes that allows us to see them under a microscope, and added to the DNA dots on the micro-/macro- array. Due to a phenomenon termed base-pairing, RNA will be “captured” to the gene it came from. After washing away all of the free RNA, we can see which RNA remains stuck to the DNA spots. Since we know which gene each spot represents, and the RNA only sticks to the gene that encoded it, we can determine which genes are turned on or off in diseases versus healthy human tissues. The genes that are expressed differently in the two tissues may be involved in causing the disease. These genes may work as markers for this kind of disease in diagnostics.

Is it possible that we could transfer this powerful technology to the field of protein study? Now, the questions are:

1. Which molecules should be used to “capture” the proteins we are interested in?

2. Could we still use the same or other technology to visualize the captured protein?

For any new technology to have an immediate and dramatic effect, it must conform to the existing technology base. In the case of protein screening, the 96-well plate-based enzyme-linked immunosorbent assay (ELISA) represents this existing technology base. The ELISA has supplanted others, becoming a major technique to detect antibody-antigen interaction with high versatility, sensitivity, and quantification, that requires little equipment and for which critical reagents are readily available.

As we have known, in our immune system B cell immunity characterized most simply by the accumulation of specific antibodies against specific foreign invading molecules (or termed antigen), has been the subject of intense biochemical and cellular study for almost a century. The practical advantage of an immunological approach to the study of protein, and cells is that, when properly exploited, the immune system can be induced into providing unique and powerful reagents antigens—antibodies.

To detect soluble (interested proteins), antibody sandwich ELISAs may be the most useful. Solid-phase reactants are prepared by adsorbing specific (capture) antibodies onto the wells of a plastic microtiter plate followed by incubation with test solutions containing antigens. Unbound antigens are washed out and a different antigen-specific antibody conjugated to an enzyme (i.e. developing antibody) is added, followed by another incubation. Unbound conjugate is washed out and a chromogenic or fluorogenic substrate is added. As the substrate is hydrolyzed by the bound enzyme conjugate, a colored or fluorescent product is generated. Finally, the product is detected visually or with a microtiter plate reader. The amount of product generated is proportional to the amount of analyze in the test mixture.

Based on the need to reduce the assay volume, a rapid evolution of the microplate has been observed. Over the last year, a variety of prototype plates of great density (1536, 3065, and 9600 wells) have been available in a limited fashion. These plates need specific robotics with very high accurate liquid handler. The instrumentation must be able to dispense precisely aqueous volumes from 10 nanoliter to 5 microliter and 1-10 nanoliter of DMSO solutions into the small wells without splattering, forming bubbles air bubbles, or clogging.

When we replace the plastic 96-well plastic plate with a glass slide, or filter (membrane), a protein/antibody micro- or macro-array can be generated. Using this new process, arrayed poly-clonal antibodies have recognized numerous antigens from fig of tissue extracts (including tumors), crude biological samples and conditional medium from cultured mammalian cells. After a cycle of binding, washing the antibody on the surface of the membrane or chip to remove unbound proteins. Immuno-complex will be specifically determined using a mixture of monoclonal antibodies, which are able to correspond their targeting antigens respectively. Following primary monoclonal antibody binding, the membrane or chip was washed to remove excess antibody solution. Finally, detection of bound antibody complex was carried out by chemiluminescence (CCD camera) or fluorescence (laser confocal scanning devices).

### APPLICATIONS

Micro- and macro-array technology of the kind are particularly suited to diagnostic application and common research laboratories because they do not merely constitute miniaturized versions of the conventional assay methods like high density micro-plate we mentioned above, and thus do not rely on the micropumps, microchannels, micrometersized reaction compartments, and other micromachined structures. Nor, in consequence, they do not rely on the measurement of submicroliter (or nanoliter) sample volumes of such tiny size that assay sensitivities are severely reduced and large statistical variations are likely to be encountered.

As this technology uses so little amount of materials that the biological hazard is easy to dispose. There is no radioactive isotopes used in array methods, as we know that isotopes are commonly used in biological research.

The development of monoclonal and poly-clonal antibody-based macro- and micro-array technology to measure antibodies, antigens and small molecules, such as drugs, ushered in a revolution in modem diagnostic medicine. Additionally, the application of antibody-array techniques to the biochemical research lab will initiate exponential growth in our understanding of human physiology, biochemistry and genetics.

a. Antibody-arrays special for protein kinesis and phosphatases are useful to study cell signal transduction pathways.

b. Antibody-array special for tumor markers will give us a global overview on the process of cancer.

c. Antibody-array special for developmental genes will let us to gain an insight into the whole picture of animal development.

d. Antibody-arrays special for cytokines, chemkines and their receptors are great tools to study various immuno-responses such as cell activation and proliferation.

e. Antibody-arrays special for virus will be used to study virus infections, including HIV.

f. Antibody-arrays special for hormones and their receptors will be used to study endocrine system under different conditions.

g. Antibody-array special for ovogenesis and spermatogenesis will be used to study human and animal generations.

What is claimed is:

1. A process for measuring antibodies, antigens and small molecules by using mono- and poly-clonal antibody-based micro- and micro-array technology, comprising the steps of:
(a) creating a reactant by adsorbing a first set of specific (capture) antibodies into a well of a plastic microtiter plate;
(b) adding a test solution into said well;
(c) incubating said test solution with said reactant;
(d) washing out a group of unbound antigens from said test solution with said reactant;
(e) incubating said test solution with said reactant without said unbound antigens;
(f) adding a second set of antibodies that is specific for conjugating to an enzyme;
(g) washing out a set of unbound conjugates from said test solution with said reactant;
(h) adding a detecting agent to said plate that is specific for a substrate formed from said enzymes; and
(i) detecting said detecting agent for determining a quantity of said substrate.

2. A process for measuring antibodies, antigens and small molecules by using mono- and poly-clonal antibody-based micro- and micro-array technology, comprising the steps of:
(a) creating a reactant by adsorbing a first set of specific (capture) antibodies onto a glass slide;
(b) adding a test solution onto said slide;
(c) incubating said test solution with said reactant;
(d) washing out a group of unbound antigens from said test solution with said reactant;
(e) incubating said test solution with said reactant without said unbound antigens;
(f) adding a second set of antibodies that is specific for conjugating to an enzyme;
(g) washing out a set of unbound conjugates from said test solution with said reactant;
(h) adding a detecting agent to said slide that is specific for a substrate formed from said enzymes; and
(i) detecting said detecting agent for determining a quantity of said substrate.

3. A process for measuring antibodies, antigens and small molecules by using mono- and poly-clonal antibody-based micro- and micro-array technology, comprising the steps of:
(a) creating a reactant by adsorbing a first set of specific (capture) antibodies into a well of a filter (membrane);
(b) adding a test solution into said filter;
(c) incubating said test solution with said reactant;
(d) washing out a group of unbound antigens from said test solution with said reactant;
(e) incubating said test solution with said reactant without said unbound antigens;
(f) adding a second set of antibodies that is specific for conjugating to an enzyme;
(g) washing out a set of unbound conjugates from said test solution with said reactant;
(h) adding a detecting agent to said filter that is specific for a substrate formed from said enzymes; and
(i) detecting said detecting agent for determining a quantity of said substrate.
